(19) United States
(12) Patent Application Publication
(10) Pub. No.: US 2021/0062179 A1
(43) Pub. Date: Mar. 4, 2021

(54) MICROSOMAL BIOREACTOR FOR SYNTHESIS OF DRUG METABOLITES

(71) Applicant: THE BOARD OF REGENTS FOR OKLAHOMA STATE UNIVERSITY, Stillwater, OK (US)

(72) Inventors: Sadagopan KRISHNAN, Stillwater, OK (US); Charuksha Thameera WALGAMA, Stillwater, OK (US); Rajasekhara Reddy NERIMETLA, Stillwater, OK (US)

(21) Appl. No.: 17/073,647

(22) Filed: Oct. 19, 2020

Related U.S. Application Data
(63) Continuation of application No. 15/552,068, filed on Aug. 18, 2017, now abandoned.
(60) Provisional application No. 62/121,105, filed on Feb. 26, 2015.

(51) Int. Cl. C12N 11/14; C12M 1/40; C12M 1/42; C12P 33/06

(52) U.S. Cl. CPC C12N 11/14; C12M 21/18; C12Y 114/00; C12P 33/06; C12M 35/02

(57) ABSTRACT

Reusable microsomal biocatalytic systems (bioreactors) constructed on carbon nanostructure modified electrodes are provided. The bioreactors comprise stable, biologically active immobilized enzymes such as human cytochromes P 450 (CYPs) and their redox partner proteins, e.g. CYP-NADPH (reduced nicotinamide adenine dinucleotide phosphate) reductases (CPR), on the carbon nanostructure surface. The immobilized enzymes may be present in liver microsomes, such as human liver microsomes (HLM) or as bactosomes, S9 fractions, etc. The bioreactors are used, for example, for synthesizing metabolites of interest from compounds such as drugs that are catabolized by the enzymes.

### CROSS REFERENCE TO RELATED APPLICATIONS

This application is a continuation of co-pending U.S. application Ser. No. 15/552,068, filed Aug. 18, 2017, which application is a 371 entry into the U.S. from PCT/US2016/019930, filed Feb. 26, 2016, which PCT application claims the benefit of U.S. Provisional Patent Application Ser. No. 62/121,105 filed on Feb. 26, 2015, and incorporates said applications by reference into this document as if fully set out at this point.

### TECHNICAL FIELD

This disclosure is related to reusable bioreactors comprising biocatalytically active microsomal enzymes immobilized on carbon nanostructure (e.g. carbon nanotubes, graphene, Buckypaper, and graphitic materials)-coated electrodes. In particular, the disclosure relates to the use of the bioractors to produce metabolites formed by the immobilized enzymes e.g. metabolites of compounds of interest such as drugs.

### BACKGROUND

Human liver membrane-bound enzymes (HLM) are subcellular fractions which contain major drug metabolizing cytochrome P450 (CYP) enzymes and their redox partner protein, CYP-NADPH reductase (CPR).^{1,2} The broad range of biocatalytic reactions catalyzed by CYP enzymes (57 isoforms of which are known in human liver), with their inherent stereoselectivity, have induced chemists to examine this unique class of enzymes for structure-function, biosensing, and catalytic applications.³⁻⁶ For new drug development in pharmaceutical industries and research laboratories, HLM are used as in vitro systems to study drug metabolism, inhibition, and drug-drug interactions.⁷ These in vitro assays use nicotinamide adenine dinucleotide phosphate (NADPH) as the electron source. The electrons derived from NADPH are mediated by CPR via its flavin adenine dinucleotide (FAD) and flavin mononucleotide (FMN) cofactors to reduce CYP enzymes in their heme iron-Fe^{III} state to heme iron-Fe^{II}, thereby facilitating dioxygen binding. Following a second electron reduction, from CPR in most cases but from cyt b₅ in some CYP catalysis, the strong oxidant that is formed, ferryloxy-CYP cation radical, can oxygenate bound drugs.

The efficacy and pharmacokinetic properties of a new drug depend on the biological activity of its metabolites formed in the liver and other organs, mainly via CYP enzyme catalyzed drug metabolism. The formation of reactive metabolites from a drug can also cause toxic effects, e.g. by damaging DNA and cellular protein-protein interactions. Hence, it is of the utmost importance to study the physicochemical and toxicity properties of the metabolites of new drugs during development, for which sufficient quantities of the metabolites must be available.^{8,9}

Arnold and co-workers have made distinguished contributions in the protein engineering of CYP enzymes to favorably tune their catalytic specificity and activity towards converting a desired substrate into products.¹⁰ Rusling et al. pioneered the CYP direct electrochemistry and electrocatalysis in films of polyions, purified CYP assembled with CYP-NADPH reductase (CPR), rat and human liver membrane-bound enzymes and genetically engineered specific CYP with CPR (“supersomes”) assembled as layer-by-layer films with polyions on electrodes.¹¹ Gilardi et al. prepared CYP-fused CPR proteins by protein engineering to enhance catalytic activity.¹² Mie et al. designed thiolated gold electrodes with hydrophobic units to immobilize supersomes for electrocatalytic applications.¹³

There is a need in the art to provide systems and methods for producing large quantities of physiologically relevant drug metabolites so that they can be adequately tested.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

The present disclosure describes the first demonstration that enzymes can be adsorbed onto carbon nanostructure-coated electrodes in bioactive form. In the case of membrane-bound enzymes, immobilization as described herein advantageously provides the enzymes with a near-in vivo environment, since other partner enzymes, cofactors, etc. are also present in the membranes. However, isolated, purified enzymes, or partner or complementary enzymes (e.g. enzymes that function in the same pathway), or even groups of isolated, purified and then recombined enzymes, may also be immobilized and used as described herein. The bioreactors are used for enhanced in vitro production of various metabolites of interest via direct enzyme electrocatalysis of compounds of interest such as drugs. Further, the bioreactors are reusable. The invention thus sets a novel direction in the design of multiuse, drug metabolizing CYP enzyme bioreactors that do not require the tedious, expensive, and time-consuming purification of CYP enzymes. The nano-bioreactors are the first of their kind to accomplish voltage-driven drug screening, drug metabolism and inhibition assays, and drug metabolite production. The bioreactors may be used, e.g. for pharmacological studies, and in biosensing and bioremediation applications, among others.

In an exemplary aspect, it is demonstrated herein that HLM, when immobilized on carbon nanostructure coated electrodes, retains its electrocatalytic capabilities and mimics its in vivo function of catalysing the conversion of compounds such as drugs into their metabolites. Carbon nanostructure-modified electrodes with adsorbed HLM can therefore be used to produce the metabolites in useful quantities. Further, the HLM-nanocarbon electrodes disclosed herein exhibit excellent stability and can be reused for multiple rounds of electrocatalysis. Thus, these reusable bioreactors represent “green” technology, e.g. for the determination of phramacokinetic properties of microsomal enzymes and for manufacturing CYP-generated metabolites.

According to an embodiment, there is provided herein a novel, reusable human liver microsomal biocatalytic system constructed on carbon nanostructure modified electrodes for green drug metabolite synthesis in an aqueous medium at room temperature. Human liver membrane-bound enzymes (HLM) were adsorbed to multiwalled carbon nanotubes coated on edge plane graphite electrodes (PGE/MWNT). Direct electron transfer between the microsomal redox proteins and the PGE/MWNT electrode was observed by cyclic and square wave voltammetry. The designed films of HLM exhibited enhanced testosterone hydroxylation when compared to HLM adsorbed on a PGE with no MWNT. The designed HLM bioreactor on PGE/MWNT surface was reusable and found to be reasonably stable with a half-life of 10 h in the electrocatalytically active oxygen reduction form. This is the first report on the successful electrocatalysis driven by HLM on carbon nanostructure electrodes and possesses immense significance in pharmaceutical industry and pharmacology research for green synthesis of drug metabolites to examine pharmacokinetic properties.

This disclosure is significant and novel in demonstrating the biocatalytic reactions of liver enzymes immobilized on high surface area nanostructure electrodes to allow design of viable bioreactors for drug metabolite synthesis.

The invention provides bioreactor devices, comprising an electrode coated with carbon nanostructured material, and one or more enzymes on the carbon nanostructured material. In some aspects, the enzymes are membrane-bound enzymes while in others the enzymes are not associated with a membrane. In some aspects, the one or more membrane-bound enzymes are present in a microsome, a bactosome or an S9 fraction. In some aspects, the one or more enzymes are liver enzymes and may be, for example, human liver enzymes. In some aspects, the one or more enzymes are drug metabolizing enzymes. In other aspects, the enzymes comprise biocatalytically active cytochromes P 450 (CYPs) and/or CYP-NADPH (reduced nicotinamide adenine dinucleotide phosphate) reductases (CPRs). In additional aspects, of the invention, the carbon nanostructured material is selected from the group consisting of single walled carbon nanotubes, multiwalled carbon nanotubes, Buckypaper and graphene nanostructures. In other aspects, the electrode is a conductive metallic or non-metallic material. In yet further aspects, the electrode is an edge-plane pyrolytic graphite electrode.

The invention also provides methods of making a bioreactor device. The methods comprise steps of coating an electrode with carbon nanostructured material, and putting one or more enzymes on the carbon nanostructured material. In some aspects, the enzymes are membrane-bound enzymes while in others the enzymes are not associated with a membrane. In some aspects, the one or more membrane-bound enzymes are present in a a microsome, a bactosome or an S9 fraction. In other aspects, the one or more enzymes are liver enzymes, and may be e.g. human liver enzymes. In further aspects, of the invention, the one or more enzymes are drug metabolizing enzymes. In yet further aspects, the enzymes comprise biocatalytically active cytochromes P 450 (CYPs) and/or CYP-NADPH (reduced nicotinamide adenine dinucleotide phosphate) reductases (CPRs). In aspects of the invention, the carbon nanostructured material is selected from the group consisting of single walled carbon nanotubes, multiwalled carbon nanotubes, Buckypaper and graphene nanostructures. In further aspects, the electrode is a conductive metallic or non-metallic material. In yet further aspects, the electrode is an edge-plane pyrolytic graphite electrode.

The invention also provides methods of producing metabolites of a compound. The first step of the method comprises i) contacting the compound with a bioreactor device comprising an electrode coated with carbon nanostructured material and one or more enzymes on the carbon nanostructured material. In some aspects, the enzymes are membrane-bound enzymes while in others the enzymes are not associated with a membrane. The step of contacting is carried out under conditions so as to permit production of metabolites of the compound by at least one of the one or more enzymes. A second step of the method comprises ii) recovering metabolites produced in the contacting step. In some aspects, the conditions include performing the step of contacting under anaerobic conditions in a physiologically compatible medium. In some aspects, the compound is a drug. In some aspects, the one or more membrane-bound enzymes are present in a a microsome, a bactosome or an S9 fraction. In other aspects, the one or more enzymes are liver enzymes, and may be e.g. human liver enzymes. In further aspects, of the invention, the one or more enzymes are drug metabolizing enzymes. In yet further aspects, the enzymes comprise biocatalytically active cytochromes P 450 (CYPs) and/or CYP-NADPH (reduced nicotinamide adenine dinucleotide phosphate) reductases (CPRs). In aspects of the invention, the carbon nanostructured material is selected from the group consisting of single walled carbon nanotubes, multiwalled carbon nanotubes, Buckypaper and graphene nanostructures. In further aspects, the electrode is a conductive metallic or non-metallic material. In yet further aspects, the electrode is an edge-plane pyrolytic graphite electrode.

The invention further provides methods of identifying metabolites of a compound produced by biocatalytic activity of one or more microsomal enzymes. The methods comprise steps of: i) contacting the compound with a bioreactor device comprising an electrode coated with carbon nanostructured material, and one or more enzymes on the carbon nanostructured material, wherein the step of contacting is carried out under conditions so as to permit production of metabolites of the compound by at least one of the one or more membrane-bound enzymes; ii) recovering metabolites produced in the contacting step; and iii) identifying the metabolites recovered in the recovering step. In some aspects, the enzymes are membrane-bound enzymes while in others the enzymes are not associated with a membrane. In some aspects, the compound is a drug. In some aspects, the one or more membrane-bound enzymes are present in a microsome, a bactosome or an S9 fraction. In other aspects, the one or more enzymes are liver enzymes, and may be e.g. human liver enzymes. In further aspects, of the invention, the one or more enzymes are drug metabolizing enzymes. In aspects of the invention, the carbon nanostructured material is selected from the group consisting of single walled carbon nanotubes, multiwalled carbon nanotubes, Buckypaper and graphene nanostructures. In further aspects, the electrode is a conductive metallic or non-metallic material. In yet further aspects, the electrode is an edge-plane pyrolytic graphite electrode. In some aspects, the one or more membrane-bound enzymes include at least one cytochrome P 450 (CYP) and the compound is a drug.

According to an embodiment there is provided a bioreactor device, comprising an electrode coated with carbon nanostructured material, and one or more enzymes on the carbon nanostructured material. According to a further embodiment, there is provided a method of making a bioreactor device, comprising coating an electrode with carbon nanostructured material, and putting one or more enzymes on the carbon nanostructured material.

According to still another embodiment, there is provided a method of producing metabolites of a compound, comprising i) contacting the compound with a bioreactor device comprising an electrode coated with carbon nanostructured material and one or more enzymes on the carbon nanostructured material, wherein the step of contacting is carried out under conditions so as to permit production of metabolites of the compound by at least one of the one or more enzymes; and ii) recovering metabolites produced in the contacting step.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings. FIG. 1 shows a schematic diagram of an exemplary electrocatalysis or testosterone by liver membrane-bound enzymes bound to carbon nanostructures.

FIGS. 2A, 2B, and 2C depict representative SEM images of A, polished bare EPG electrode; B, coated MWNT on the EPG electrode; and C, HLM adsorbed onto the MWNT modified EPG electrode.

FIG. 3 depicts cyclic voltammograms of a, EPG/PL; b, EPG/HLM; c, EPG/MWNT/HLM; and d, EPG/MWNT electrodes under anaerobic conditions in phosphate buffer pH 7 at 25° C.; scan rate 0.3 V s⁻¹.

FIG. 4 depicts an example of a plot of square wave voltammograms of a, EPG/HLM; b, EPG/MWNT/HLM; c, EPG/MWNT; and d, EPG electrodes in anaerobic pH 7 buffer solution, amplitude 60 mV and frequency 30 Hz at 25° C.

FIG. 5 depicts a plot of rotating disc catalytic oxygen reduction voltammograms of (a) EPG/MWNT/HLM, (b) EPG/HLM, (c) EPG/MWNT, and (d) EPG/PL films in saturated oxygen, phosphate buffer, pH 7 at 25° C., 300 rpm rotation rate, scan rate 0.3 V s⁻¹.

FIG. 6 shows HPLC chromatograms of 100 μM standard testosterone and 6β-hydroxytestosterone in phosphate buffer pH 7, at 25 ° C.

FIG. 7. Reuse of carbon nanostructure-modified electrodes for electrocatalysis. Exemplary HPLC chromatograms of 250 μM testosterone after repeated rounds of 1 h of bulk electrolysis using the same EPG/MWNT/HLM electrodes (chromatograms a-d) or EPG/HLM electrodes (chromatogram e) at −0.6 V vs Ag/AgCl. The experiments were performed in phosphate buffer (pH 7.0) under saturated oxygen conditions at 25° C. For chromatograms b-d, a fresh testosterone solution was added before each experiment to evaluate the reusability of the EPG/MWNT/HLM electrodes.

FIG. 8 shows a representation of an exemplary calibration curve showing peak area vs concentration of standard 6β-hydroxytestosterone.

FIG. 9 contains an exemplary plot of an amperometric (i-t curve) assessing the catalytic oxygen reduction stability of EPG/MWNT/HLM vs Ag/AgCl electrodes at an applied potential of −0.6 V in phosphate buffer, pH 7.0, saturated oxygen, 25° C.

FIG. 10. Schematic representation of a bioreactor.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

Reusable biocatalytic systems (bioreactors, bioelectrodes) constructed on carbon nanostructure modified electrodes are provided. The bioreactors comprise catalytically active enzymes immobilized on a carbon nanostructure surface. The enzymes are capable of acting on and modifying a substrate to form metabolites of the substrate. The bioreactors are used, for example, for synthesizing metabolites of interest from compounds such as drugs that are catabolized by the enzymes. In some aspects, the enzymes are associated with a membrane (“membrane-bound enzymes”). In other aspects, the enzymes are isolated and/or purified prior to immobilization and are thus not associated with a membrane. However, even isolated and purified enzymes may be reintroduced into a membrane (e.g. a membrane in which they are not found in nature, such as a synthetic membrane or a membrane from a species in which they do not otherwise occur), prior to immobilization on the bioreactor. Exemplary enzymes include cytochrome P 450 (CYP) enzymes and their redox partner proteins CYP-NADPH (reduced nicotinamide adenine dinucleotide phosphate) reductases (CPR), e.g. from human liver. The reusable bioreactors are stable with a half-life of at least about 10 h in the electrocatalytically active oxygen reduction form. They do not require expensive cofactors and simply utilize voltage as the driving force to catalyze bio-reactions. FIG. 1 contains a schematic illustration of an exemplary electrocatalysis by liver membrane-bound enzymes bound to carbon nanotube-modified electrodes.

In some aspects, the enzymes that are immobilized on the bioreactor are not associated with a membrane. Rather, they are enzymes that have been isolated, purified or partially or substantially isolated and/or purified. The enzymes may be isolated from a natural source (e.g. from organ or other preparations) or they may be recombinant enzymes generated in an expression system, e.g. a bacterial, insect, plant or mammalian expression system. By substantially isolated and/or purified, we mean that the enzymes are largely (e.g. at least about 75%) free of other macromolecules such as proteins, nucleic acids, lipids and carbohydrates, but may still be associated with e.g. buffer or media components, cofactors, ions, etc., or even with other small molecules which do not impact the activity of the enzyme.

As used herein “membrane-bound enzymes” refers to catalytically active enzymes that are bound to (e.g. associated with, embedded in, covalently or non-covalently bonded to, etc.) a membrane. In some aspects, the membrane is a double layer of lipids that is a portion of or that mimics the membranes found in living organisms. In one aspect, the membrane-bound enzymes are present in microsomes, i.e. vesicle-like artifacts re-formed from pieces of the endoplasmic reticulum (ER) when eukaryotic cells are broken-up in a laboratory setting, and which contain one or more enzymes capable of acting on at least one substrate. The membrane-bound enzymes may be microsomal fractions which are obtained by methods that are known in the art, for example, by homogenization of tissue, followed by differential centrifugation to concentrate the membrane-bound enzymes and separate them from other cellular debris. Membrane-bound enzymes may be made from a variety of sources, e.g. liver, lung, heart, esophagus and other organs such as mitochondria, etc. In some aspects, non-enzyme proteins or polypeptides may also be included in the membranous constructs of the invention, either adventitiously or purposefully. In other aspects, the membrane preparations are free of proteins or polypeptides that are not enzymes, or at least are not enzymes of interest.

In addition, the membrane-bound enzyme preparations utilized in the practice of the invention may be synthetic (artificial) or semi-synthetic in nature. For example, fully artificial membranes or similar structures may be utilized. Exemplary artificial membranes are generally formed from lipids, and include, for example, liposomes, i.e. synthetic “sacs” which are generally formed from phospholipids and which may also contain additional lipid and/or protein moieties. Alternatively, the artificial membranes may be sheet-like in structure. One or more enzymes capable of acting on at least one substrate of interest are associated with the artificial membrane, generally by being embedded in the membrane, although surface attached enzymes and enzymes located within a liposome are also contemplated. When synthetic membranes are used, the enzymes attached to or embedded in the membrane may be isolated and/or purified from a natural source, or may have been produced via recombinant techniques as described below.

Other sources of membrane-bound enzymes are also contemplated. For example, microsomes prepared and isolated and/or purified from one species may be used to entrap or embed enzymes from a different species e.g. bacterial- or insect based membranes may contain human enzymes , either by adding the human enzymes after preparation of the membranes, or by synthesizing recombinant human enzymes in bacterial or insect cell culture expression systems, etc. For example, membrane-bound enzymes containing specific types or amounts of CYPs may be prepared from E.coli or Sf9 insect cell culture via heterologous expression of enzymes of interest. Examples include but are not limited to bactosomes, which are bacterial membranes containing e.g. the human cytochrome P450s co-expressed with human NADPH-cytochrome P450 reductase. S9 fractions may also be utilized. Alternatively, suitable membrane-bound protein preparations are readily commercially available. Examples include but are not limited to BD Scientific Superomes™, Corning® Supersomes™, etc.

Membrane-bound enzymes that are immobilized on a carbon nanoparticle coated electrode as described herein may be or may comprise one or more subcellular fractions derived from an area of interest, e.g. from the endoplasmic reticulum of liver. The fractions that are used may be from any suitable species and are generally from mammals, e.g. from humans or other primates, or from any other mammal of interest, including but not limited to companion pets (dogs, cats, horses, etc.), animals raised as live stock (e.g. cattle, sheep, goats, etc.) or other animals (e.g. rats, mice, rabbits, guinea pigs, pigs, etc.), and others. The immobilized fractions may originate from any species for which it is desired to investigate the metabolism of one or more compounds (e.g. a drug or drugs, or any other xenobiotic) and/or to generate metabolites of the compound(s). In addition, the immobilization of fractions or extracts containing enzymes of interest from non-mammalian species is also encompassed, including but not limited to various birds, fish, reptiles, plants, insects, fungi, protozoa, bacteria, etc.

The immobilized fraction may be of any suitable type. For example, it may be or comprise pooled fractions from several (2 or more) individuals (e.g. from at least 2 but as many as 10, 25, 50, 75, or 100 or more individuals); or may be from a single individual of interest. Further, the fractions may from an individual or individuals with a particular trait of interest, e.g. known to carry a genetic mutation or marker of interest, known to have a particular disease or condition, or known to exhibit one or more phenotypic characteristic, or known to be of a specific gender or age group, or combinations of these criteria, or for any other reason. In addition, other types of fractions may also be immobilized as described herein e.g. liver S9 fractions, liver cytosolic fractions, etc. In some aspects, the enzymes that are immobilized on the bioreactors of the invention are from specific populations e.g. lung membrane-bound enzymes from smokers and/or non-smokers, liver enzymes from healthy subjects vs those with liver disease, etc. All such enzymes may be used in the practice of the present invention. In some aspects, the membrane-bound enzymes that are employed are liver membrane-bound enzymes, e.g. human liver membrane-bound enzymes. Pooled fractions may be characterized (e.g. for Km and Vmax). Enzymes associated or present in liver microsomes or bactosomes, as well as other membrane-bound or isolated forms of drug metabolizing enzymes or chemical catalysts, may also be immobilized and used as described herein. Enzymes that may be present in the fractions include but are not limited to: cytochromes P450 (CYP) (e.g. CYP1A1/2, CYP2A6, CYP2C8, CYP2C9, CYP2C19, CYP2D6, CYP2E1, CYP3A4/5, CYP3A4/5, CYP4A11, etc.); flavin monooxygenases (FMOs), glutathione transferases (GSTs), monamine oxidases (MAOs), sulfurotransferases (SULTs), uridine glucuronide transferases (UGTs), and other similar classes of monooxygenases, enzymes, and redox partners, etc.

In some aspects of the invention, what is immobilized on the bioreactor is an S9 fraction. S9 fractions are the product of an organ tissue homogenate used in biological assays. The S9 fraction is most frequently used in assays that measure the metabolism of drugs and other xenobiotics and is defined by the U.S. National Library of Medicine's “IUPAC Glossary of Terms Used in Toxicology” as the “Supernatant fraction obtained from an organ (usually liver) homogenate by centrifuging at 9000 g for 20 minutes in a suitable medium; this fraction contains cytosol and microsomes.”

In other aspects, the enzymes that are utilized (whether membrane-bound or not) are recombinant, e.g. are genetically engineered enzymes which are produced, e.g. by cloning cDNA of an enzyme of interest into a suitable vector, and then using the vector to produce the recombinant enzyme, using techniques that are known in the art. The recombinant enzyme may or may not be identical in primary amino acid sequence to the parent enzyme, as changes to the sequence may be made. However, the recombinant form generally retains at least about 75, 80, 85, 90, 95% or more identity with the parent enzyme of interest. Similarly, the level of activity of the recombinant enzyme is generally at least about 75, 80, 85, 90, 95% or more of the level of the parent enzyme, and the recombinant may exhibit 100% or even more of the level of activity of the parent enzyme, i.e. the recombinant enzyme may be more active than the native (e.g. wildtype), parent enzyme. Other forms of the enzymes that are used in the practice of the invention are also encompassed, e.g. various mutants such as substitution and truncation mutants (either natural or made via genetic engineering), as well as chimeras, etc. The recombinant enzymes may be incorporated into a membrane e.g. by being synthesized in an expression system that produces them in a membrane compartment, or by being synthesized and isolated and then incorporated into or entrapped within a membrane.

The enzymes that are present on the bioreactors of the invention may have any of a variety of activities, examples of which include but are not limited to: cleavage and/or formation of covalent chemical bonds, addition or removal of functional groups to/from molecules (e.g. methyl groups, sulfates, carboxyl groups, H atoms, etc.), activation or inactivation of molecules, etc.

### Making a Bioreactor Device

The bioreactors of the invention are made by selecting a suitable solid substrate that is capable of conducting an electric current, and putting one or more types of nanostructured carbon onto a surface of the substrate. In some aspects, the substrate is an electrode and will generally be referred to as an “electrode” herein. However, the invention encompasses the use of other suitable substrates that are capable of conducting an electrical current, but which may not technically be termed “electrodes”. The electrode may be of any suitable composition and/or type. The one or more types of nanostructured carbon is put onto the surface of the substrate by being adsorbed, absorbed, impregnated into, coated onto or otherwise adhered to the surface by any suitable method that results in retention of sufficient material on the surface to receive membrane-bound enzymes, as described below.

The material that is put onto the electrode is nanostructured carbon, e.g. is formed from carbon nanoparticles. A “carbon nanostructure” refers to an artificially composed carbon structure having at least one dimension that is on a nanometer scale, e.g. that is less than about 100 nanometers. Exemplary carbon nanostructures include but are not limited to: graphene sheets or bent or folded graphene, nanotubes (e.g. armchair, zig-zag and chiral configurations) which may be singlewalled or multiwalled, nanocones, nanohorns, fullerenes, various negatively curved nanostructures, nanofibers, nanoribbons, nanostars, and the like, and composites thereof such as sulfur composites.

To coat the electrode with nanostructured material, the electrode is generally exposed to or contacted with a liquid in which the nanostructured material has been dispersed, e.g. as a slurry. Dispersion is performed e.g. by a technique such as ultrasonication or other high shear mixing technique which deagglomerates the carbon nanomaterial. The concentration of nanostructured material in the liquid is generally in the range of from about 0.1 to about 3.0 mg mL⁻¹, and is preferably about 1.0 mg mL⁻¹. The liquid may be aqueous or non-aqueous (organic) Exemplary liquids include but are not limited to dimethylformamide (DMF), as n-methylpyrrolidone (NMP), toluene, phenyl ethyl alcohol, dichloromethane, ethanol, isopropyl alcohol, hexane, and all other aqueous solvents, polymeric, surfactant, ionic liquids, and DNA based solutions.

The electrodes which are used in the practice of the invention are, for example, edge plane pyrolytic graphite electrodes, and all other conductive metallic and non-metallic surfaces and materials can be used.

The carbon nanostructure solutions in suitable solvents are applied to an outer surface of the electrode and allowed to dry coat by leaving it for several hours at room temperature or heating e.g. at about 60° C. in an oven or by using any suitable technique as desired, e.g. by ultrasonic spray, by dipping the electrode in the dispersion, by “painting” the dispersion onto the electrode, and by other chemical and physical methods. Generally, the carbon nanostructured coating is applied to a thickness of from about 25 nm to about 1 micron or more, e.g. about 25, 50, 75, 100, 125, 150, 175, 200, 225, 250, 500 and 1000 nm or more from dispersions of concentrations prepared in suitable solvents as described above (non-aqueous and aqueous solvents). Following application, the coating is dried.

Once the coating is dry, the nanostructured surface is washed well in deionized water and then exposed to or contacted with a solution or dispersion comprising enzymes in order to put the enzymes onto the nanostructured material, e.g. in the form of a file or coating. The enzymes are put onto (applied to) the surface of the substrate by being adsorbed to, absorbed to, impregnated into, coated onto or otherwise adhered to, attached to or associated with the surface by any suitable method that results in retention of sufficient membrane-bound enzymes to form a bioreactor device as described herein. The enzymes are typically in an aqueous, physiologically compatible medium such as phosphate buffer, pH 7.4, at a total protein concentration of from about 2 to about 20 mg/mL, which is kept at low temperature, e.g. less than about 10° C., e.g. about 4-5° C. during adsorption. The solution is left in contact with the nanostructured surface for a period of time that is sufficient for the enzymes to attach or adsorb to the surface e.g. for from about 15 minutes to about one hour, e.g. for about 30 minutes. The exposure or contact may be performed e.g. immersing the electrode in a microsome solution, or rinsing the electrode with the solution, pipetting the solution directly onto the electrode, spraying a solution of membrane-bound enzymes, etc. The electrochemically active concentration of membrane-bound enzymes (known from cyclic voltammetry) that is deposited on the nanostructure-coated surface typically ranges from about 25 to about 500 picomoles per cm² of geometric electrode surface, and the total amount of surface area that contains adsorbed membrane-bound enzymes is generally from about 0.1 to about 0.2 cm². Preferably, following adsorption and prior to use, the electrode is stored, e.g. at about 4° C. in an aqueous buffer or in water, for a period of time ranging from, for example, about 8 to 24 hours or even longer, e.g. up to about 2 days.

In some aspects, bioreactor production is scaled up for industrial use. For example, design of carbon nanomaterial coated electrodes with geometric area of 5 to 50 cm² or even larger with appropriate engineering of the reactor design.

A schematic representation of a bioreactor of the invention is presented in FIG. 10. In this figure, surface 15 of electrode 10 is coated with nanostructured carbon layer 20. Microsomal layer 30 is in turn adsorbed onto nanostructured carbon layer 20. Microsomal layer 30 comprises surface accessible microsomal enzymes 40.

### Use Of The Bioreactor

The bioreactors described herein are used for a variety of purposes, including but not limited to as a research tool for various types of studies such as cytochrome P450 inhibition studies metabolic stability, cytochrome P450 phenotyping, metabolite characterization, metabolite production, slowly metabolizing drugs, bioremediation processes, toxicity and pharmacological assays, biosensors for small and large molecule screening, industrial waste treatment, and any other related enzyme based catalytic and sensing applications, etc.

Electrocatalysis of a compound or compounds of interest is performed by exposing the immobilized microsomal enzymes or contacting the immobilized microsomal enzymes with one or more compounds or substances of interest, for which it is desired to produce metabolites thereof, or to ascertain whether or not the microsomal enzymes generate metabolites of the compound. Generally, this is accomplished by immersing the electrode in a solution comprising the compound e.g. a biocompatible solution such as saline, phosphate buffer, so-called Good's buffer such as MOPS (3-(N-morpholino) propanesulfonic acid) and HEPES (4-(2-hydroxyethyl)-1-piperazineethanesulfonic acid), etc. Generally, the solution is buffered at a pH in the range of from about 7.2 to about 7.6, e.g. about 7.4.

Analysis and recovery of metabolites produced by the action of the enzymes on the bioreactor may be accomplished by any suitable technique, many of which are known in the art. For example, production may be detected or monitored by HPLC, other types of chromatography, by NMR, etc.; and recovery, isolation and/or purification may be performed by various combinations of precipitation, centrifugation, filtration, chromatography (e.g. size exclusion and affinity chromatography), or by other known techniques.

The electrocatalysis of a wide variety of compounds may be performed using the bioreactors described herein. Exemplary compounds include but are not limited to: various drugs and pharmaceuticals, various so-called “neutraceuticals”, pollutants, fertilizer components, compounds used in manufacturing (e.g. those used in manufacturing plastics, paints, resins, solvents, etc.), various toxins, insecticides, and other substrates that are converted to products by enzymes, etc. Any compound that is metabolized by microsomal enzymes such as CYPs, or which is suspected of being metabolized by microsomal enzymes such as CYPs, may be analyzed as described herein.

Exemplary compounds that may be investigated and/or used as substrates for the production of metabolites as described herein included but are not limited to: phenacetin, coumarin, bupropion, paclitaxel, tolbutamide, (S) mephenytoin, dextromethorphan, chlorzoxazone, midazolam, testosterone, lauric acid, any drug, e.g. those used in clinical trials, etc.

Exemplary metabolites that can be investigated and/or produced using the bioreactors and methods described herein include but are not limited to: 6β-hydroxy testosterone, acetaminophen, 7-hydroxycoumarin, hydroxybupropion, 6α-hydroxypaclitaxel, hydroxytolubutamide, 4′-hydroxymephenytoin, dextrorphan, 6-hydroxychlorzoxazone, 1′-hydroxymidazolam, 6β-hydroxytestosterone, 12-hydroxydecanoic acid, methyl p-tolylsulfideh, 7-hydroxycoumarin glucuronide, etc.

### EXAMPLES

10 μL of 1 mg/ml⁻¹ MWNT dispersion in dimethylformamide (DMF) (obtained by 4 h ultrasonication in a water bath) was dry coated on Parallel Gap Electrodes (PGE) (geometric area 0.2 cm²). Following this, 20 μL of HLM was placed on the PGE/MWNT surface and adsorbed for 30 minutes at 4° C. The electrodes were rinsed in water and stored overnight at 4° C. before using for electrochemical and electrocatalytic experiments. The overnight storage provided better film stability than the electrodes used immediately after adsorbing HLM.

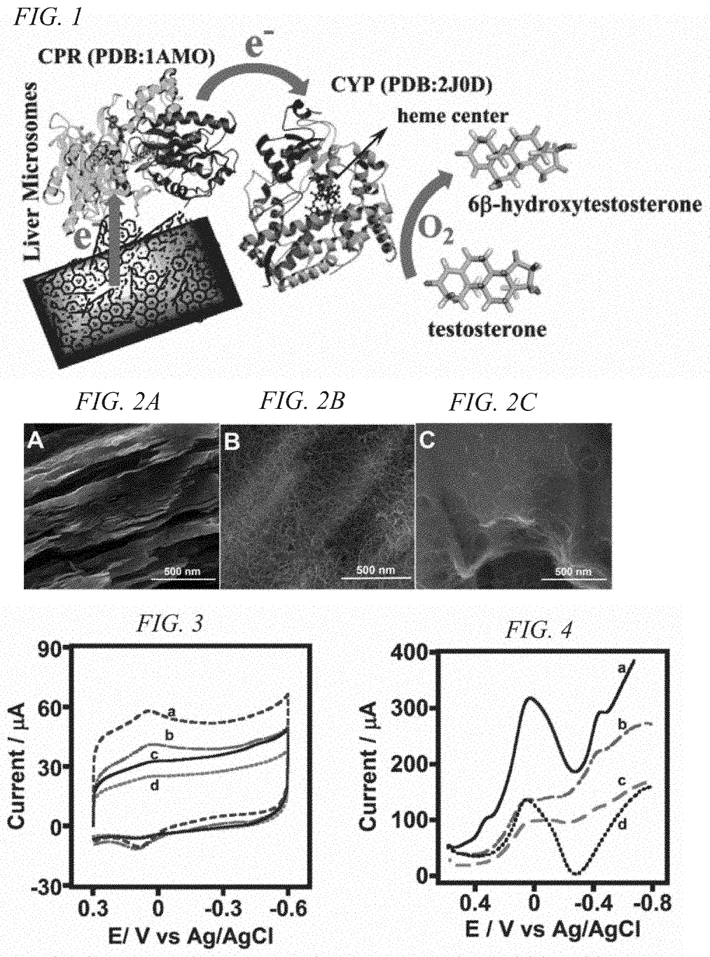

The surface morphologies of PGE, PGE/MWNT, and HLM coated on PGE/MWNT electrodes were characterized by scanning electron microscopy (SEM) as shown in FIGS. 2A, 2B, and 2C. The characteristic surface defects of PGE were covered by MWNT upon coating and subsequent adsorption of HLM resulted in the formation of a new uniformly coated layer around MWNT, confirming the immobilization of HLM on PGE/MWNT surface (FIGS. 2A, 2B, and 2C).

The cyclic voltammograms of the designed liver membrane-bound enzymes adsorbed on PGE/MWNT electrodes displayed a redox pair at a formal potential of −0.46 V vs Ag/AgCl (FIGS. 3b&c), which is in agreement with the formal potential of microsomal CPR film. Control electrodes of PGE/PL (phospholipid, PL) and PGE/MWNT (FIGS. 3a&d) did not show any peak in the CPR potential region, but exhibited a redox pair at a positive formal potential region (˜80 mV vs Ag/AgCl), which is characteristic of the edge plane surfaces of pyrolytic graphite and MWNT. Furthermore, square wave voltammetry (SWV) data confirmed the results obtained from cyclic voltammetry (FIG. 4). As can be seen in FIG. 4, the higher sensitivity of SWV over cyclic voltammetry led to significantly larger currents for the microsomal CPR peak at the negative potential region. Taken together, the cyclic voltammetry and SWV results confirmed the successful attainment of direct electronic communication between membrane-bound enzymes and MWNT-modified electrodes.

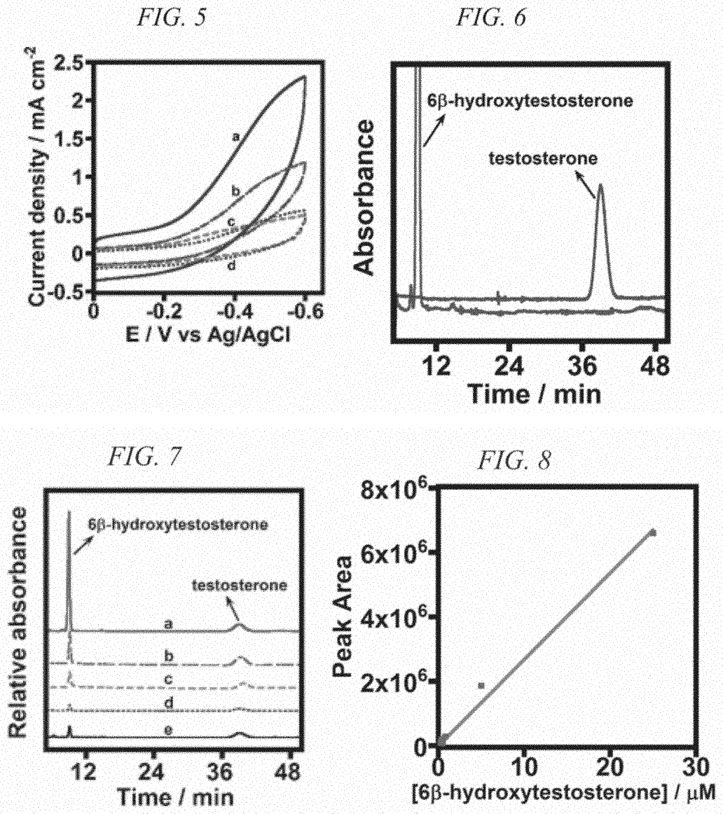

The electrocatalytic oxygen reduction currents catalyzed by the designed films of PGE/MWNT/HLM was greater than the PGE/HLM by about 2.0-times (FIG. 5, curves a and b). On the other hand, the control PGE/PL and PGE/MWNT electrodes in the absence of adsorbed membrane-bound enzymes showed small reduction currents arising from the electrocatalytic property of edge planes of pyrolytic graphite and those present in MWNT (FIG. 5, curves c and d).¹⁴

In addition to the enhanced oxygen reduction currents, the PGE/MWNT/HLM electrodes exhibited electrode-driven bioactivity in converting testosterone to 6β-hydroxytestosterone, which is characteristic of CYP enzymes present in HLM. The biocatalytic property of microsomal films on PGE/MWNT/HLM and PGE/HLM electrodes was studied by bulk electrolysis at −0.6 V vs Ag/AgCl in the presence of oxygen in phosphate buffer (pH 7.0) containing dissolved testosterone and by analyzing the reaction mixture using high performance liquid chromatography (HPLC). The identification of the reactant and product in the chromatograms were accomplished by running standard solutions of testosterone and 6β-hydroxytestosterone under similar conditions (FIG. 6). CYP 2C19, 2C9, and 3A4 present in HLM have been shown to hydroxylate testosterone.¹⁵

FIG. 7 shows the chromatograms of 6β-hydroxy testosterone product formation from testosterone conversion electrocatalyzed by PGE/MWNT/HLM (chromatogram a) or by PGE/HLM (chromatogram e). The product formation in the testosterone electrocatalysis confirms the role of CYP enzymes present in HLM in catalyzing the testosterone conversion in PGE/MWNT/HLM and PGE/HLM electrodes. The reusability of PGE/MWNT/HLM electrodes was examined by replenishing a fresh testosterone solution following the first electrolysis and by continuing the electrolysis reaction under the applied potential of −0.6 V vs Ag/AgCl (FIG. 7, chromatograms b-d).

Since the observed direct electrochemistry is of microsomal reductase, the testosterone hydroxylation by microsomal CYP enzymes in the HLM is suggested to involve electron mediation by reductases from the electrode to CYP-heme centers, similar to that reported before. By obtaining the calibration plot of standard 6β-hydroxytestosterone (FIG. 8), it was possible to quantitate that 2.2 nmol of metabolite was formed by PGE/MWNT/HLM and 0.4 nmol of metabolite was formed by PGE/HLM electrodes per unit PGE geometric area (in cm²). This suggests a 5-fold enhancement in metabolite amount by HLM immobilized on MWNT-modified electrodes.

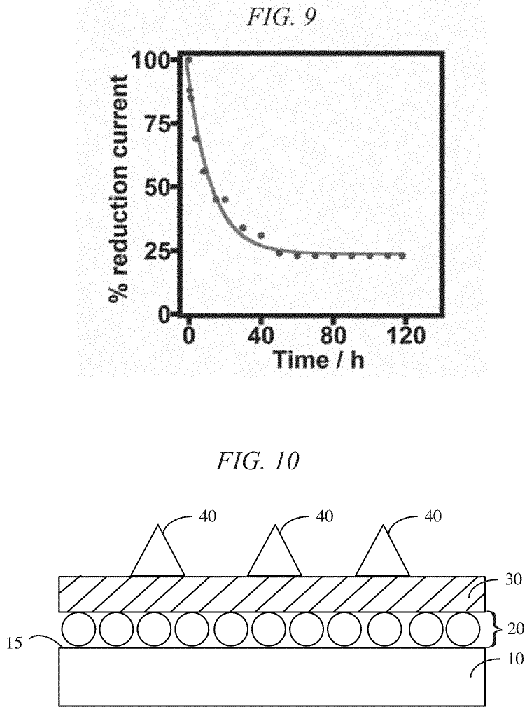

FIG. 7, chromatogram b shows that ˜45% of initial product yield was obtained upon the reuse of PGE/MWNT/HLM electrodes. Subsequent reusing of the electrodes further decreased the amount of metabolites to 25% (2^{nd} reuse, FIG. 7, chromatogram c) and 9% (3^{rd} reuse, FIG. 7, chromatogram d) of the initial metabolite yields. Hence, the question is to identify the cause of decreasing product yields with number of reuse: FIG. 9 presents the film stability of liver membrane-bound enzymes coated on the designed, catalytically superior PGE/MWNT electrode in the presence of oxygen examined by chronoamperometry.

The stability of HLM films on PGE/MWNT electrodes does not appear to affect the catalytic yields significantly, as within 4 h, only about ˜20% loss in catalytic currents is noted. Moreover, the half-life of PGE/MWNT/HLM electrode was found to be 10 h (FIG. 9), which is sufficient to complete the biocatalysis with 3-times reusability of the electrodes as demonstrated in FIG. 7. Therefore, the decrease in yield with number of reuse is suggested to be due to the hydrogen peroxide formed from the electrochemical reduction of oxygen in the electrolysis solution and its detrimental effect to the microsomal membranes via lipid peroxidation and the possible inactivation of bound CYP enzymes. Detailed understanding of the underlying mechanisms needs further investigations.

Nevertheless, stability of electocatalytic enzyme films has been a huge challenge to achieve. In the case of designed complex liver microsomal films on nanostructure electrodes, the bioactive film stability could be expected to be even more challenging. Despite this presumption, reasonably good stability for the designed films of HLM on EPG/MWNT electrodes were observed, which suggests favorable secondary interactions of microsomal membranes with carbon nanotubes to keep the membrane-bound proteins intact (FIG. 9).

In summary, an embodiment shows the successful development of electrochemical liver microsomal bioreactors on carbon nanostructured electrodes for the first time. Additionally, the observed direct electrochemical communication between the microsomal proteins and MWNT-modified electrodes, direct electrocatalysis, sufficient catalytic stability, and reusability features suggest a new direction in the design of practically viable enzyme bioreactors, not requiring purified enzymes, for green fine chemicals syntheses and biosensing applications.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described. Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)−(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein.

Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

### REFERENCES

(1) von Bahr, C.; Groth, C.-G.; Jansson, H.; Lundgren, G.; Lind, M.; Glaumann, H. Clin. Pharmacol. Ther. 1980, 27, 711-725.
(2) Ortiz de Montellano, P. R. 3rd Ed., Cytochrome P450, Kluwer/Plenum, New York, 2005.
(3) Guengerich, F. P. J. Biochem. Mol. Toxicol. 2007, 21,163-168.
(4) Li, A. P. Drug Discov. Today Technol. 2005, 2, 179-185.
(5) Lin, J. H. ; Lu, A. Y. H. Pharmacol. Rev. 1997, 49, 403-449
(6) Krishnan, S.; Bajrami, B.; Hvastkovs, E. G. ; Choudhary, D.; Schenkman, J. B.; Rusling, J. F. Anal. Chem. 2008, 80, 5279-5285.
(7) Wienkers, L. C. ; Heath, T. G. Nat. Rev. Drug Discov. 2005, 4, 825-833.
(8) Park, B. K. ; Boobis, A.; Clarke, S. et al. Nat. Rev. Drug Discov. 2011, 10, 292-306.
(9) Deavall, D. G. ; Martin, E. A.; Horner, J. M. ; Roberts, R. J. Toxicol. vol. 2012, Article ID 645460, 13 pages, 2012. doi: 10.1155/2012/645460
(10) Jung, S. T.; Lauchli, R.; Arnold, F. H. Curr. Opin. Biotechnol. 2011, 22, 809-817.
(11) Krishnan, S.; Schenkman, J. B.; Rusling, J. F. J. Phys. Chem. B 2011, 115, 8371-8380.
(12) Dodhia, V. R. ; Sassone, C.; Fantuzzi, A.; Nardo, G. D. ; Sadeghi, S. J.; Gilardi, G. Electrochem. Commun. 2008, 10, 1744-1747.
(13) Mie, Y.; Suzuki, M.; Komatsu, Y. J. Am. Chem. Soc. 2009, 131, 6646-6647.
(14) Banks, C. E. ; Compton, R. G. Analyst 2006, 131, 15-21.
(15) Yamazaki, H.; Shimada, T. Arch. Biochem. Biophys. 1997, 346, 161-169.

What is claimed is:

1. A bioreactor device, comprising
an electrode coated with carbon nanostructured material, and
one or more enzymes on the carbon nanostructured material.

2. The bioreactor device of claim 1, wherein the one or more enzymes are i) membrane-bound enzymes; or ii) enzymes that are not associated with a membrane.

3. The bioreactor device of claim 2, wherein the membrane-bound enzymes are present in a microsome, a bactosome or an S9 fraction.

4. The bioreactor device of claim 1, wherein the enzymes are liver enzymes.

5. The bioreactor device of claim 4, wherein the liver enzymes are human liver enzymes.

6. The bioreactor device of claim 1, wherein the one or more enzymes are drug metabolizing enzymes.

7. The bioreactor of claim 1, wherein the enzymes comprise biocatalytically active cytochromes P 450 (CYPs) and/or CYP-NADPH (reduced nicotinamide adenine dinucleotide phosphate) reductases (CPRs).

8. The bioreactor device of claim 1, wherein the carbon nanostructured material is selected from the group consisting of single walled carbon nanotubes, multiwalled carbon nanotubes, Buckypaper and graphene nanostructures.

9. The bioreactor device of claim 1, wherein the electrode is a conductive metallic or non-metallic material.

10. The bioreactor device of claim 1, wherein the electrode is an edge-plane pyrolytic graphite electrode.

11. A method of making a bioreactor device, comprising
coating an electrode with carbon nanostructured material, and
putting one or more enzymes on the carbon nanostructured material.

12. The method of claim 11, wherein the one or more enzymes are i) membrane-bound enzymes; ii) enzymes that are not associated with a membrane.

13. The method of claim 12, wherein the one or more membrane-bound enzymes are present in a microsome, a bactosome or an S9 fraction.

14. The method of claim 11, wherein the one or more enzymes are liver enzymes.

15. The method of claim 14, wherein the liver enzymes are human liver enzymes.

16. The method of claim 11, wherein the one or more enzymes are drug metabolizing enzymes.

17. The method of claim 11, wherein the enzymes comprise biocatalytically active cytochromes P 450 (CYPs) and/or CYP-NADPH (reduced nicotinamide adenine dinucleotide phosphate) reductases (CPRs).

18. The method of claim 11, wherein the carbon nanostructured material is selected from the group consisting of single walled carbon nanotubes, multiwalled carbon nanotubes, Buckypaper and graphene nanostructures.

19. The method of claim 11, wherein the electrode is a conductive metallic or non-metallic material.

20. The method of claim 11, wherein the electrode is an edge-plane pyrolytic graphite electrode.

21. A method of producing metabolites of a compound, comprising
i) contacting the compound with a bioreactor device comprising
an electrode coated with carbon nanostructured material and
one or more enzymes on the carbon nanostructured material,
wherein the step of contacting is carried out under conditions so as to permit production of metabolites of the compound by at least one of the one or more enzymes; and
ii) recovering metabolites produced in the contacting step.

22. The method of claim 21, wherein the conditions include performing the step of contacting under anaerobic conditions in a physiologically compatible medium.

23. The method of claim 21, wherein the one or more enzymes include at least one cytochrome P 450 (CYP) and the compound is a drug.

24. The method of claim 21, further comprising a step of identifying metabolites produced in said step of contacting.
